(19) United States
(12) Patent Application Publication
(10) Pub. No.: US 2013/0131509 A1
(43) Pub. Date: May 23, 2013

(54) SYSTEMS AND METHODS FOR BREAST IMAGING

(71) Applicant: General Electric Company, Schenectady, NY (US)

(72) Inventors: Tzachi Rafaeli, Shlmenit (IL); Ira Micah Blevis, Zicron Yaakov (IL); Yaron Hefetz, Kibbutz alonim (IL)

(73) Assignee: General Electric Company, Schenectady, NY (US)

(21) Appl. No.: 13/683,947

(22) Filed: Nov. 21, 2012

Related U.S. Application Data
(60) Provisional application No. 61/562,853, filed on Nov. 22, 2011.

(51) Int. Cl. A61B 10/00; A61B 5/00; G01T 1/161

(52) U.S. Cl. CPC A61B 10/0041; G01T 1/161; A61B 5/004
USPC 600/436; 250/394

(57) ABSTRACT

Systems and methods for breast imaging are provided. One includes a gantry, a first nuclear medicine detector mounted to the gantry and having a multi-bore collimator coupled thereto, and a second nuclear medicine detector mounted to the gantry and having a multi-bore collimator coupled thereto. The first and second nuclear medicine detectors are configured to be independently titled with respect to a breast therebetween.

### CROSS REFERENCE TO RELATED APPLICATIONS

This application claims priority to and the benefit of the filing date of U.S. Provisional Application No. 61/562,853, filed Nov. 22, 2011, the subject matter of which is hereby incorporated by reference in its entirety.

### BACKGROUND OF THE INVENTION

The subject matter disclosed herein relates generally to nuclear medicine (NM) imaging systems, and more particularly to methods and systems for breast imaging with NM imaging systems, in particular Molecular Breast Imaging (MBI).

Mammography imaging is commonly used for the detection of breast cancer. Specifically, mammography imaging is used to detect lesions within the breast. Typically, the lesion is detected using three-dimensional imaging techniques. As such, a location and depth of the lesion can be determined from the image. The depth of the lesion aids, for example, in guiding a biopsy needle during extraction of a lesion sample for pathology.

However, some women cannot be effectively tested because of dense breasts and/or implants. Accordingly, these women may be tested using nuclear single photon imaging. Such imaging only provides two-dimensional images of the lesion having no depth information. When the depth of the lesion is unknown, guiding a biopsy needle is difficult and the chance of missing the lesion with the needle is increased, which is often high. As a result, a large number of samples may have to be taken, thereby causing pain and discomfort to the patient.

### BRIEF DESCRIPTION OF THE INVENTION

In one embodiment, a breast imaging system is provided that includes a gantry, a first nuclear medicine detector mounted to the gantry and having a multi-bore collimator coupled thereto, and a second nuclear medicine detector mounted to the gantry and having a multi-bore collimator coupled thereto. The first and second nuclear medicine detectors are configured to be independently titled with respect to a breast therebetween.

In another embodiment, a breast imaging system is provided that includes a gantry, a first nuclear medicine detector mounted to the gantry and having a multi-bore collimator coupled thereto and a second nuclear medicine detector mounted to the gantry and having a multi-bore collimator coupled thereto. The first and second nuclear medicine detectors are configured to be independently titled with respect to a breast therebetween and the multi-bore collimators coupled to the first and second nuclear medicine detector have slanted bores, with the bores of the multi-bore collimator coupled to the first nuclear medicine detector slanted at a different angle than the bores of the multi-bore collimator coupled to the second nuclear medicine detector.

In yet another embodiment, a method for breast imaging is provided. The method includes positioning a pair of opposing nuclear medicine detectors at an oblique angle to each other, wherein the nuclear medicine detectors include a multi-bore collimator coupled thereto. The method also includes maintaining a position of a breast between the nuclear medicine detectors and acquiring nuclear medicine imaging data based on emissions from an agent injected into the breast. The method further includes computing a depth of a lesion in the breast based on a relative difference in the location of the lesion imaged by each of the nuclear medicine detectors.

### BRIEF DESCRIPTION OF THE DRAWINGS

FIG. 1 is a schematic block diagram of an exemplary nuclear medicine (NM) imaging system constructed in accordance with various embodiments.

FIG. 2 is a diagram of a detector configuration in accordance with one embodiment.

FIG. 3 is a diagram illustrating apparent images acquired by different detectors.

FIG. 4 is a diagram illustrating a detector configuration in one position in accordance with one embodiment.

FIG. 5 is a diagram illustrating a detector configuration in another position in accordance with one embodiment.

FIG. 6 is a diagram of a detector configuration in accordance with another embodiment.

FIG. 7 is a diagram illustrating different detector arrangement for acquiring different views in accordance with various embodiments.

FIG. 8 is a diagram of a detector configuration in accordance with another embodiment.

FIG. 9 is a diagram of a detector configuration in accordance with an embodiment showing a biopsy guiding device.

FIG. 10 is another diagram of a detector configuration in accordance with an embodiment showing a biopsy guiding device.

FIGS. 11-14 are diagrams of different support structures in accordance with various embodiments.

### DETAILED DESCRIPTION OF THE INVENTION

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Also as used herein, the phrase “reconstructing an image” is not intended to exclude embodiments in which data representing an image is generated, but a viewable image is not. Therefore, as used herein the term “image” broadly refers to both viewable images and data representing a viewable image. However, many embodiments generate, or are configured to generate, at least one viewable image.

Various embodiments described herein provide systems and methods for Nuclear Medicine (NM) imaging of breasts, also referred to herein as Molecular Breast Imaging (MBI). Various embodiments generally include a dual-head nuclear breast imaging system having a pair of detector heads that are independently rotatable about one or more axis to provide different angular orientations of the detector heads relative to a breast. By practicing various embodiments, depth information may be provided, such as to estimate the depth of a lesion within a breast.

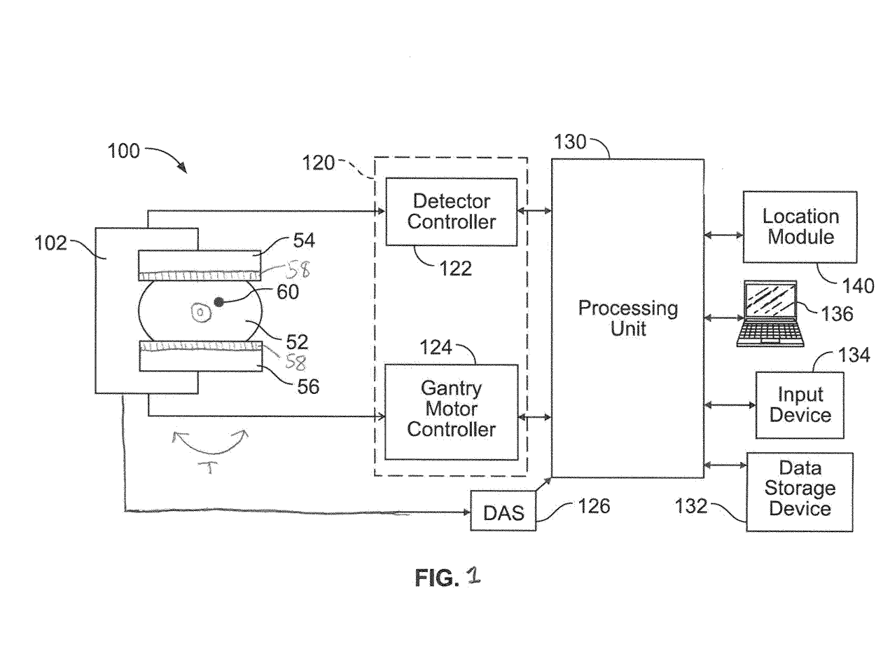

FIG. 1 is a schematic block diagram of an NM imaging system 100 including first and second detectors 54 and 56 mounted on a gantry 102. The first and second detectors 54 and 56 are configured in some embodiments as a pair of imaging detectors 54 and 56 that are each independently and individually controllable, including movement of the detectors 54 and 56 (e.g., rotation about one or more axis). For example, the first and/or second detectors 54 and 56 may be titled along an axis transverse to a breast 52 (as illustrated by the arrow T) or may be tilted along an axis generally perpendicular to the body of an imaged patient as described in more detail herein. It should be noted that the detectors 54 and 56 may be titled in the same or different directions and at the same or different angles.

The first and second detectors 54 and 56 are arranged and operate to provide two-dimensional imaging of the breast 52. The first and second detectors 54 and 56 are illustrated as planar single photon imaging detectors, however, other configurations may be provided. In various embodiments, the first and second detectors 54 and 56 may be formed of cadmium zinc telluride (CZT) tiles or may be any type of two-dimensional pixelated detector. The detectors 54 and 56 also include collimators 58 coupled thereto on a detection surface of the detectors 54 and 56, which are illustrated as parallel hole collimators 58. However, other types of collimators may be provided, such as diverging, converging, pinhole, cone-beam, fan-beam or slanted collimators, among others.

Each detector 54 and 56 captures a two-dimensional image that may be defined by the x and y location of a pixel and a detector number. At least one of the detectors 54 and 56 may change orientation relative to a stationary or movable gantry 102. Because the detectors 54 and 56 are registered, features appearing at a given location in one detector 54 and/or 56 can be correctly located and the data correlated in the other detector 54 and/or 56.

Each of the detectors 54 and 56 has a radiation detection face that is directed towards a structure of interest, for example a lesion 60, within the breast 52. The radiation detection faces are covered by the collimator 58 as described above. An actual field of view (FOV) of each of the detectors 54 and 56 may be directly proportional to the size and shape of the respective imaging detector, or may be changed using the collimator 58.

A motion controller unit 120 may control the movement and positioning of the gantry 102 and/or the detectors 54 and 56 with respect to each other to position the breast 52 within the FOVs of the imaging detectors 54 and 56 prior to acquiring an image of the breast 52. The controller unit 120 may have a detector controller 122 and gantry motor controller 124 that may be automatically commanded by a processing unit 130, manually controlled by an operator, or a combination thereof. The gantry motor controller 124 and the detector controller 122 may move the gantry 102 and the detectors 54 and 56 individually with respect to the breast 52, with the distance between the detectors 54 and 56 and the orientations thereof registered by the controller 120 and used by the processing unit 130 during data processing. In some embodiments, motion is manually achieved and the controller 120 is replaced with scales or preferably encoders for measuring at least the distance between the detectors 54 and 56, as well as the orientation and and/or the compression force exerted by at least one of the detector 54 and/or 56 on the breast 52.

In operation, the detectors 54 and 56 and gantry 102 remain stationary after being initially positioned, and imaging data is acquired, as discussed below, which may include acquiring emission data or gamma radiation activity count data from an agent, such as a radiopharmaceutical or radioactive tracer, injected within the patient (e.g., injected into the breast). The imaging data may be combined and reconstructed into a composite image comprising 2D images and depth information.

A Data Acquisition System (DAS) 126 receives analog and/or digital electrical signal data produced by the detectors 54 and 56 and decodes the data for subsequent processing in the processing unit 130. A data storage device 132 may be provided to store data from the DAS 126 or reconstructed image data. An input device 134 also may be provided to receive user inputs and a display 136 may be provided to display reconstructed images.

The NM imaging system 100 also includes a location module 140 configured to perform one or more methods described herein, for example, to determine the depth of the lesion 60 in the breast 52. Although FIG. 1 shows the location module 140 as a separate module, it should be appreciated that the location module 140 can also be a program, software, or the like stored on a computer readable medium to be read by the NM imaging system 100.

In operation, the detectors 54 and 56 are capable of being independently or individually rotated to different angles to provide various images or views of the breast 52, which in various embodiments, results in the detectors 54 and 56 being positioned in a non-parallel arrangement with respect to each other. In various embodiments, the distance between the two detectors 54 and 56 may be changed to accommodate breasts with different sizes and to immobilize the breast for the duration of data acquisition by applying light pressure (e.g., less than a pressure applied during an x-ray mammography exam). The distance between near faces of the two collimators 58 is registered automatically or manually. In one embodiment, one of the detectors moves while the other remains stationary, for example, the upper detector 54 moves toward the lower detector 56 (as viewed in FIG. 1) to immobilize the breast 52 therebetween. Thus, the detectors 54 and 56 are used to apply an immobilizing force to the breast 52. Accordingly, in one embodiment, the breast 52 is positioned between the detectors 54 and 56 and at least one detector is translated to lightly compress and/or maintain the position of the breast 52 between the detectors 54 and 56. It should be noted that the compression of the breast 52 shown in the various figures is exaggerated for illustration. Thus, the distance between the faces of the two collimators 58 in various embodiments is equal to the thickness of the slightly compressed breast, which is registered by the detectors 54 and 56 and may be used by a data analysis program.

The detectors are then used to provide image data of the breast 52 and one or more lesions 60, for example a breast cancer tumor, within the breast 52. As can be seen, the lesion 60 may be located some depth within the breast, and thus at a different distance from each detector, thereby creating different image data in each of the detectors 54 and 56. As described in more detail below, the images from the detectors 54 and 56 may be used to determine a position, as well as a depth of the lesion 60 within the breast 52. For example, the depth of the lesion 60 may be calculated based on simple geometry as described below and then used for determining a direction for insertion of a biopsy needle into the breast 52. It should be noted that the various embodiments may be used to determine the three-dimensional (3D) locations of more than one lesion 60 within the breast 52.

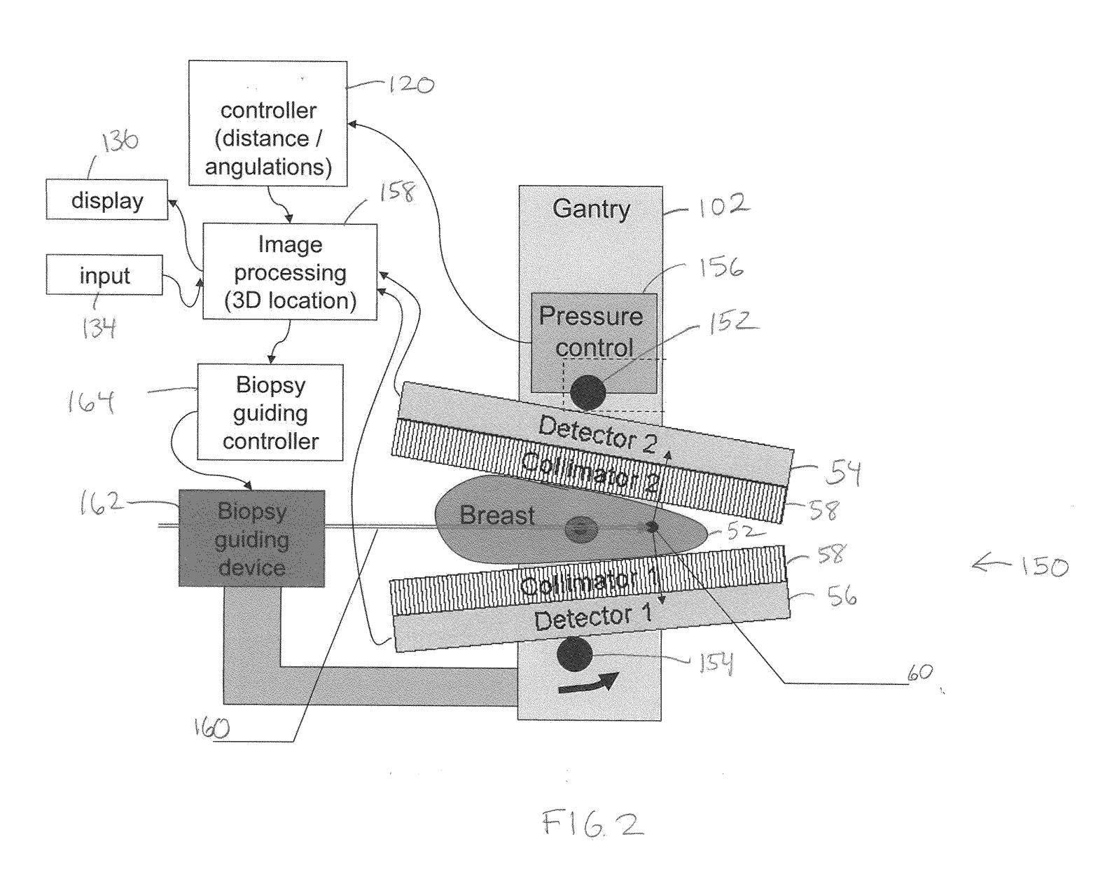

Thus, various embodiments provide the detectors 54 and 56 in slanted configurations for MBI. For example, FIG. 2 illustrates a slanted detector configuration 150 for imaging the breast 52 and which may be used to determine the depth of the lesion 60 within the breast 52. As can be seen, the detectors 54 and 56 are configured to rotate about pivot points 152 and 154 (as illustrated by the arrow). The detectors 54 and 56 are rotated to be titled at opposite angles to each other in the illustrated embodiment, which may be the same or different amount of rotation or tilting for each detector 54 and 56. For example, as can be seen, the detectors 54 and 56 may be titled such that the detectors 54 and 56 are closer together at the portion of the breast 52 in the area by or proximate the lesion 60 and farther apart at the portion of the breast 52 where there is no lesion 60. Thus, the breast 52 may be compressed more in the area closer to the lesion 60 such that the detectors 54 and 56 are moved in closer proximity to the lesion 60. However, the lesion 60 may also be in the area that is not as compressed. The movement of the detectors 54 and 56 towards and away from the breast 52, which in one embodiment is translation of the detector 54, is controlled in part by the detector controller 122 and gantry motor controller 124 (both shown in FIG. 1), in combination with a pressure controller 156. Thus, in one embodiment, the pressure controller 156 controls the amount of pressure applied to the breast 52 by the detector 54 to immobilize the breast 52 between the detector 54 and the detector 56, wherein the detector 54 is stationary along the gantry 102.

Data acquired by the detectors 54 and 56 is provided to an image processing module 158 and/or the location module 140 (shown in FIG. 1). The location module 140 is used to determine the 3D location of the lesion 60 within the breast 52, which may be used to guide a biopsy needle 160 into the breast 52 toward the lesion 60. The movement of the biopsy needle 160 may be provided by a biopsy guiding device 162 controlled by a biopsy guiding controller 164. The biopsy guiding device 162 and the biopsy guiding controller 164 may be provided using any suitable guiding mechanisms or apparatus and may receive lesion location information prior to and/or during insertion (e.g., location feedback information) of the biopsy needle 160 into the breast 52.

In one embodiment, operation may be provided, for example, as follows:

a. Each of the two opposing detectors 54 and 56 is fitted with a multi-bore, parallel-hole collimator 58 having bores at 90 degrees to the detectors 54 and 56.

b. The two detectors 54 and 56 (with attached collimators 58) are positioned at an angle with respect to each other (e.g., arranged in a non-parallel relationship to each other with the breast 52 therebetween).

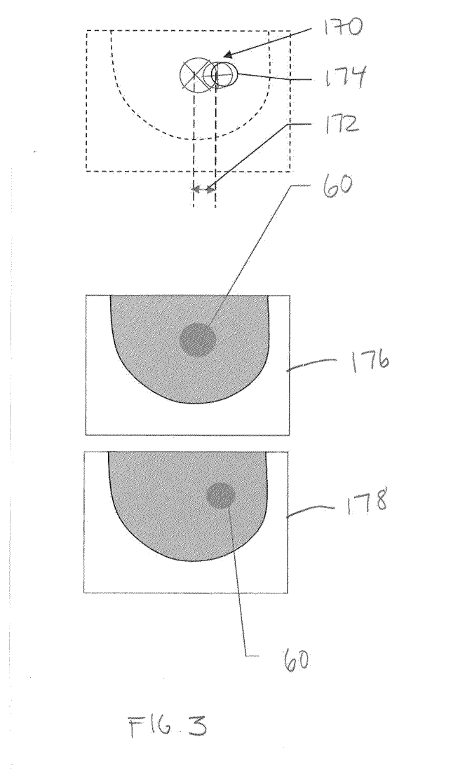

c. The depth of the lesion 60 in the breast 52 is computed by the relative difference 170 in the location of the lesion 60 appearing on the detectors 54 and 56 (e.g., using a data subtraction process) as shown in FIG. 3. The difference in apparent location depends on the distance from the center of the breast 52. Thus, FIG. 3 shows the difference 172 in apparent lesion location/size from an actual lesion location 174. For example, the apparent location of the lesion 60 as acquired by the detector 54 is shown in the image 176 and the apparent location of the lesion 60 as acquired by the detector 56 is shown in the image 178.

d. The biopsy guiding device 162 is used to guide the biopsy needle 160 to the location of the lesion 60 based on the calculation of the location in 3D space. The biopsy guiding device 162 in various embodiments is attached to the structure holding the detectors 54 and 56 (e.g., the gantry 102) and in known coordinates with respect to the detectors 54 and 56 (and thus to the breast 52 and lesion 60).

e. The detectors 54 and 56 can swivel, such that, for example, a configuration of parallel detectors may be used for “non-biopsy” imaging. However, in other embodiments only one detector can or does swivel.

It should be noted that in various embodiments, the large opening (namely the region where the detectors 54 and 56 are farther apart) is “outwards” to allow easier access of the biopsy tool (e.g., larger spacing between the detectors 54 and 56 proximate the location of the biopsy tool), which includes the biopsy needle 160 and the biopsy guiding device 162. It also should be noted that the depth of the lesion 60 may be determined using any suitable method.

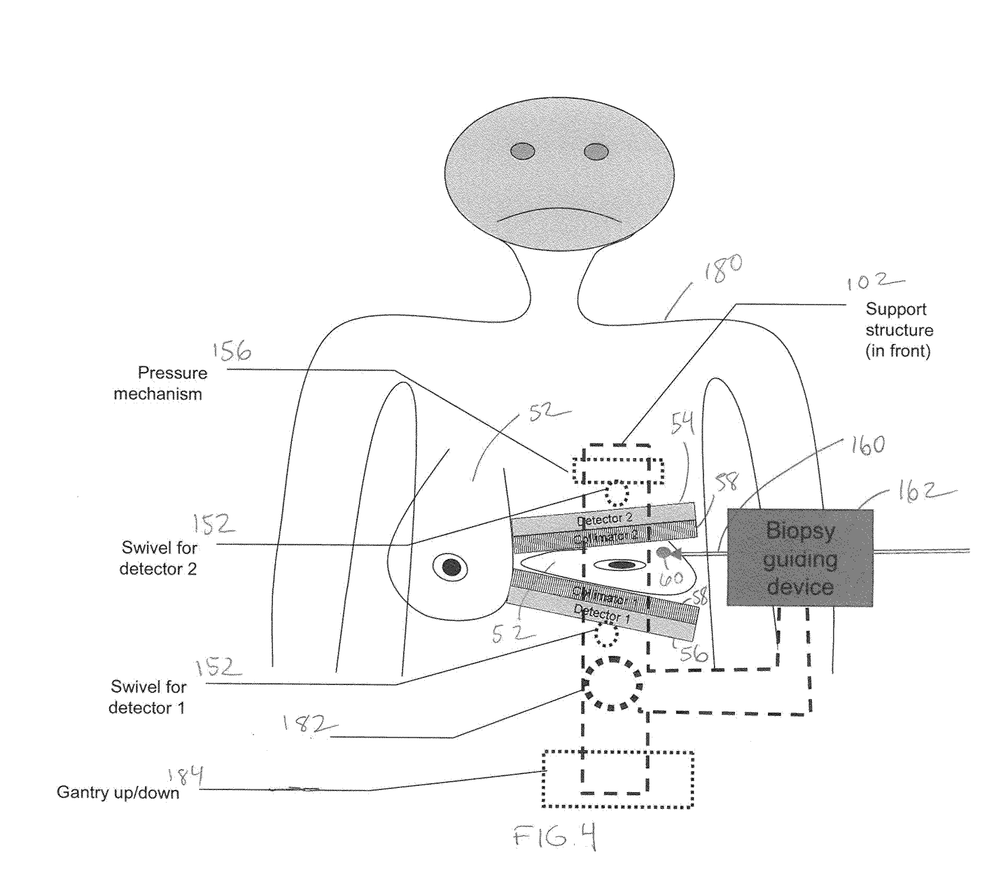

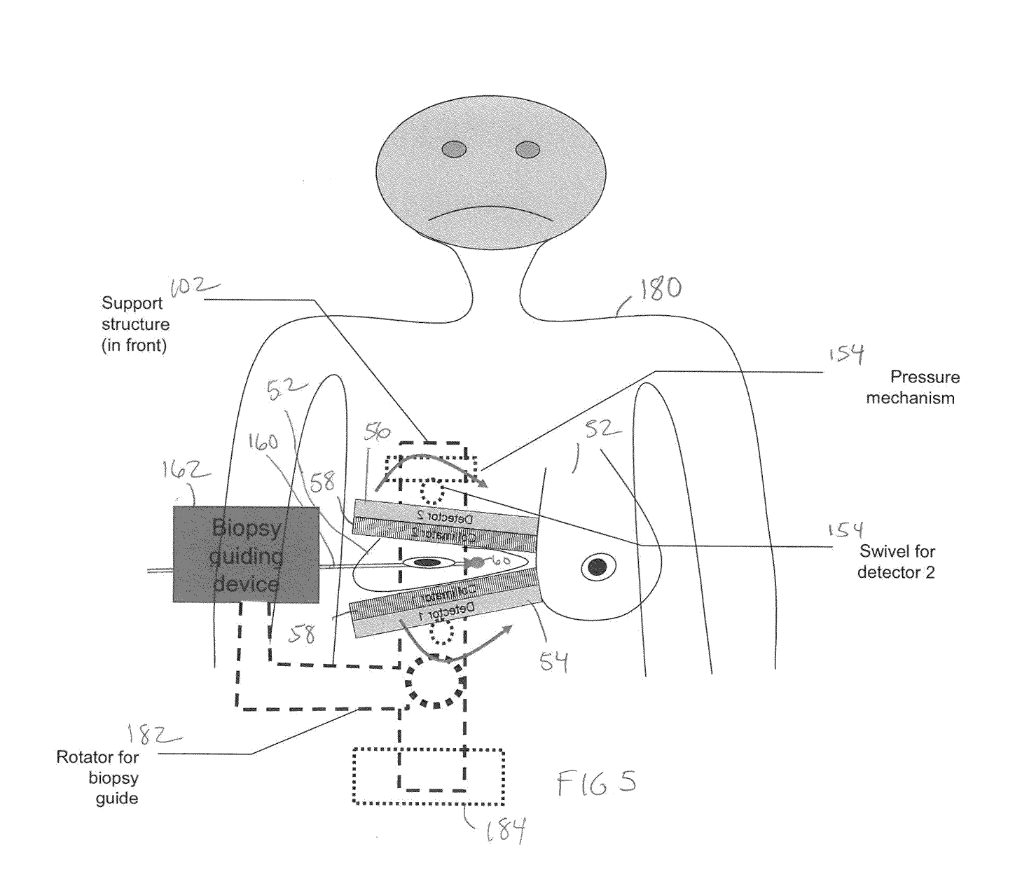

Thus, a left breast 52 of a patient 180 may be imaged and/or the depth of the lesion 60 determined for biopsy as shown in FIG. 4. The detectors 54 and 56 then may be rotated about the gantry 102 and the right breast 52 of the patient 180 may be imaged and/or the depth of the lesion 60 determined for biopsy as shown in FIG. 5. As can be seen, the location of the detectors 54 and 56 are reversed in the two configurations of FIGS. 4 and 5, namely the upper detector in one configuration is the lower detector in the other configuration. It should be noted that a rotator 182 also may be provided for rotating the biopsy guiding device 162 and a gantry motor 184 provided to move the detectors 54 and/or 56 along the gantry 102 (which may be controlled by the gantry motor controller 124 shown in FIG. 1). It should be noted that the amount of rotation and tilting of the detectors 54 and 56 may be adjusted or varied as desired or needed. For example, the relative tilting and direction of tilting of the detectors 54 and 56 may be the same or different when imaging each of the breasts 52, which in some embodiments is based on the location of a suspected lesion 60. Thus, the detectors 54 and 56 may be symmetrically or asymmetrically positioned (e.g., having different tilting angles) with the breast 52 therebetween, which likewise may be asymmetrically positioned, such as compressed more on one side than the other as described herein. For example, the detectors 54 and 56 may be obliquely angled wherein the tilt angles are the same or different for each of the detectors 54 and 56.

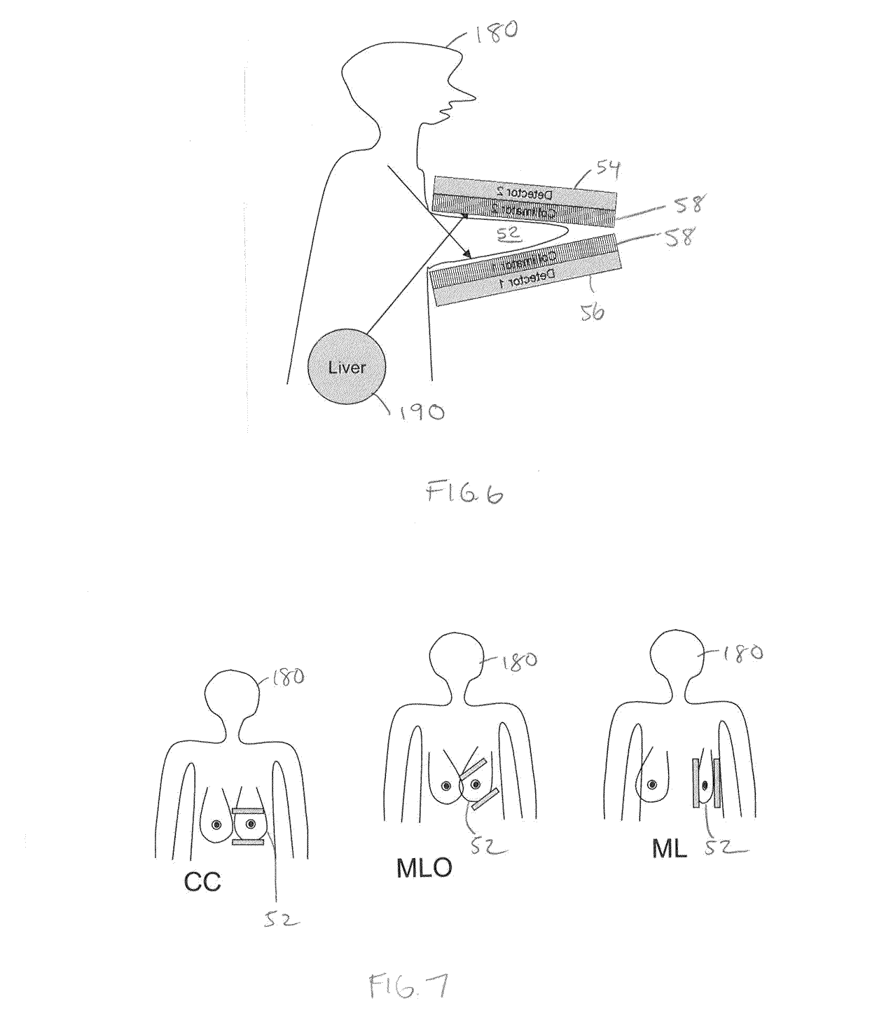

Variations and modifications are contemplated. For example, as shown in FIG. 6, the detectors 54 and 56 may be slanted towards and/or away from the body of the patient 180 such that the detectors 54 and 56 are positioned closer together towards the front or back of the breast (as viewed in FIG. 6) instead of towards the left and right of the breast (as viewed in FIGS. 4 and 5). Thus, the detectors 54 and 56 may be rotated or tilted in a plane parallel to the coronal plane of a person (e.g., rotated in a plane parallel to a front of the person or transverse to the breast 52) or tilted in a plane parallel to the sagittal plane of a person (e.g., rotated in a plane perpendicular to the front of the person). However, the detectors 54 and 56 may be configured to tilt in different directions and combinations thereof.

It should be noted that in some embodiments, one or more different positions of the detectors 54 and 56 may be provided, including, for example, a cranio-caudal (CC) position, a mediolateral (ML) position and/or a mediolateral oblique (MLO) position as shown in FIG. 7 to obtain different projections or views. In one embodiment, when using the detector configuration shown in FIG. 6, the ML and/or MLO position is used such that only the lungs are in the field or view of the detectors 54 and 56 and not other organs, such as the liver 190. Additionally, it should be noted that in addition to rotating the detectors 54 and 56, tilting of one or both of the detectors 54 and 56 may be provided such that that detectors 54 and 56 may be aligned in a parallel or non-parallel relationship.

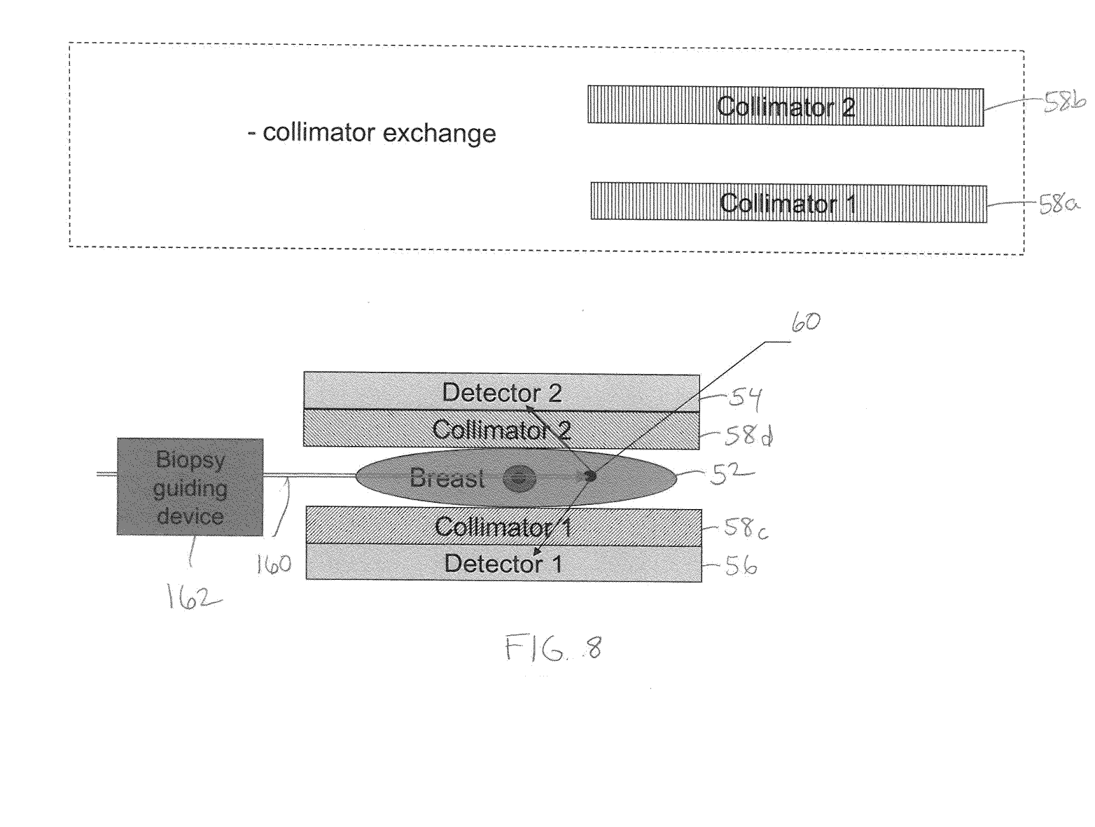

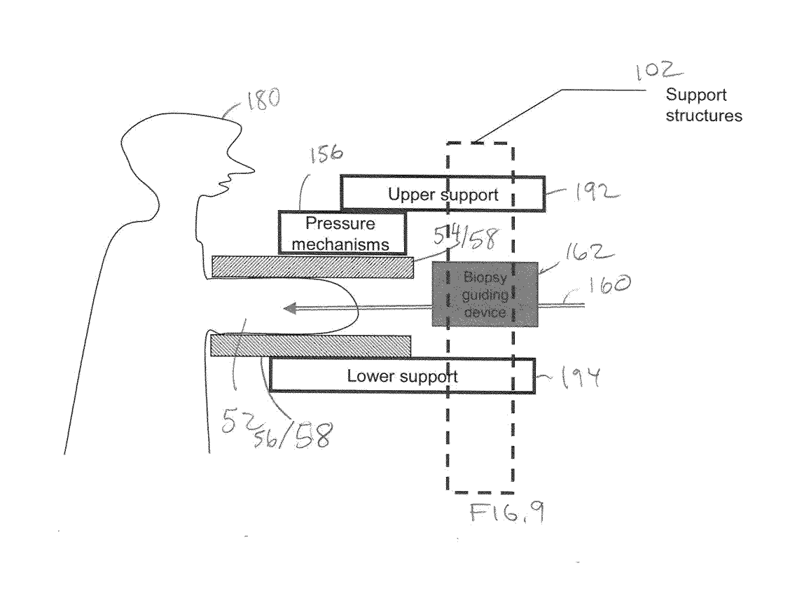

Other variations are contemplated. For example, the multi-bore, parallel-hole collimators 58a and 58b may be replaced with multi-bore, slanted-hole collimators 58c and 58d as shown in FIG. 8. It should be noted that the detectors 54 and/or 56 having the collimators 58c and 58d coupled thereto may be rotated and tilted similar to configuration shown in FIG. 2 or may be maintained generally parallel to each other as shown in FIG. 1. It should be noted that the angle of the bores in the collimators 58c and 58d may be the same and/or different and may be angled in the same and/or different direction. Also, the angles of the bores within each of the collimators 58c and 58d may be angled similarly or may have varying slant angles relative to the detection face of the detectors 54 and 56. Thus, the bores of the collimators 58c and 58d may be obliquely angled with the slant angles being the same or different within or between each of the collimators 58c and 58d.

In the illustrated embodiment, operation may be provided, for example, as follows:

a. Each of the two opposing (and substantially parallel) detectors 54 and 56 is fitted with a multi-bore, parallel-hole, slanted collimator 58c and 58d, respectively.

b. The angulations of the collimators 58c and 58d are different, for example, the bores in one collimator are obliquely or oppositely angled with respect to the bores in the other collimator.

c. The depth of the lesion 60 in the breast 52 is computed by the relative difference in the location of the lesion 60 as appearing on the detectors 54 and 56. The difference in apparent location in this embodiment is linear with the distance from the center of the breast 52.

d. The biopsy guiding device 162 is used to guide the biopsy needle 160, which may be manual, semi-automatic, or automatic, to the location of the lesion 60 based on the calculation of the location in 3D space. The biopsy guiding device 162 in various embodiments is again attached to the structure holding the detectors 54 and 56 (e.g., the gantry 102) and in known coordinates with respect to the detectors 54 and 56 (and thus to the breast 52 and lesion 60). The biopsy guiding device 162 may be, for example, a manual biopsy guide with channels for insertion of the biopsy needle 160, a stereotactic tool, a guided biopsy tool, or a robotically controlled device, among others.

The collimators 58c and 58d are exchangeable. For example, the set of collimators 58a and 58b with bores at 90 degrees to the detectors may be used for “non-biopsy” imaging or swiveled as described herein.

It should be noted that only one of the collimators may be exchangeable or switchable in various embodiments. For example, the fixed collimator may be at 90 degrees to the detector. It also should be noted that the collimators 58c and 58d may be arranged similar to FIG. 6 such that the slanted bores are angled towards or away from the body of the patient 180, however the detectors 54 and 56 may be generally parallel in some embodiments.

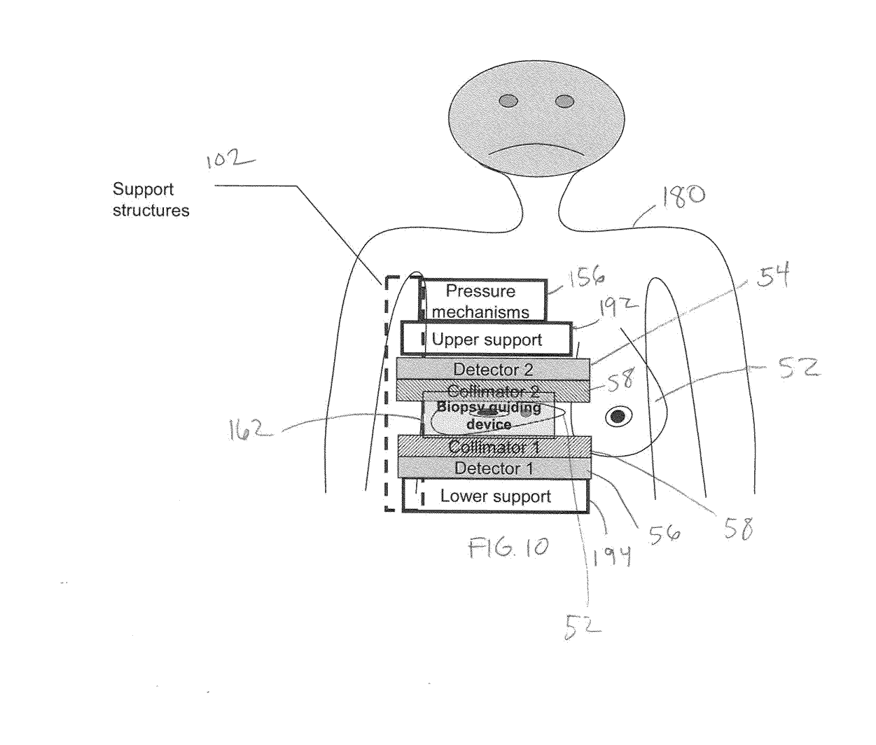

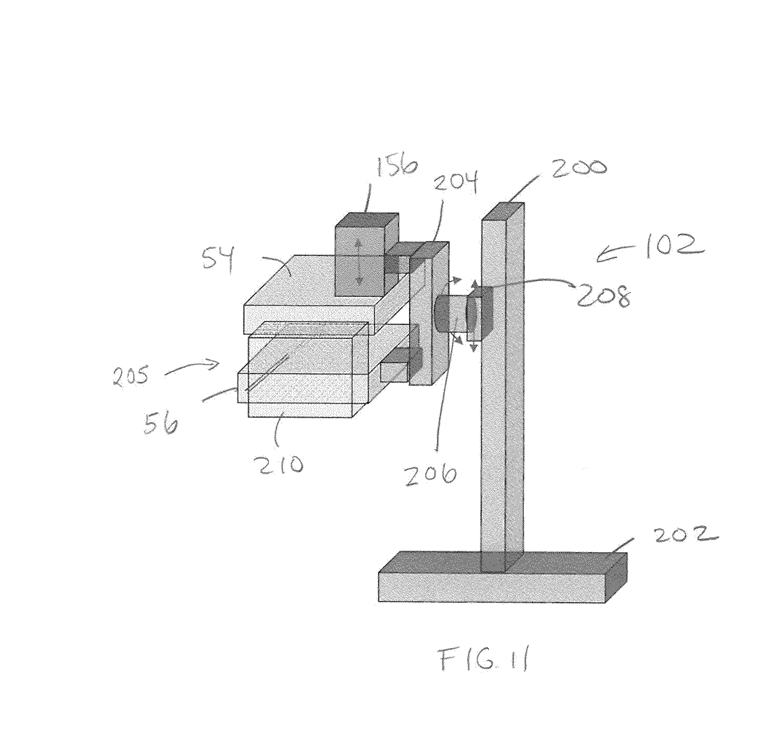

In some embodiments, for example, as shown in FIGS. 9 and 10, a frontal biopsy guide arrangement may be provided that also may include support members 192 and 194, illustrated as top and bottom supports. Thus, the biopsy needle 160 may be inserted through the front region of the breast 52 instead of through one of the sides of the breast 52. It should be noted that different supporting arrangements may be provided for the biopsy device, such as two bars or support arms on the side of the gantry 102 or in a “C-arm” type configuration. It also should be noted that if a biopsy guide is provided that is a matrix of holes then the guide may be the support structure.

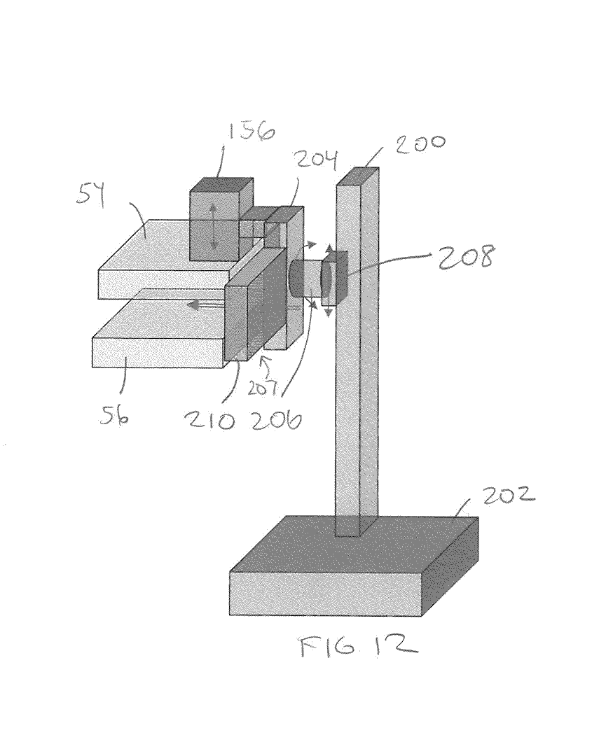

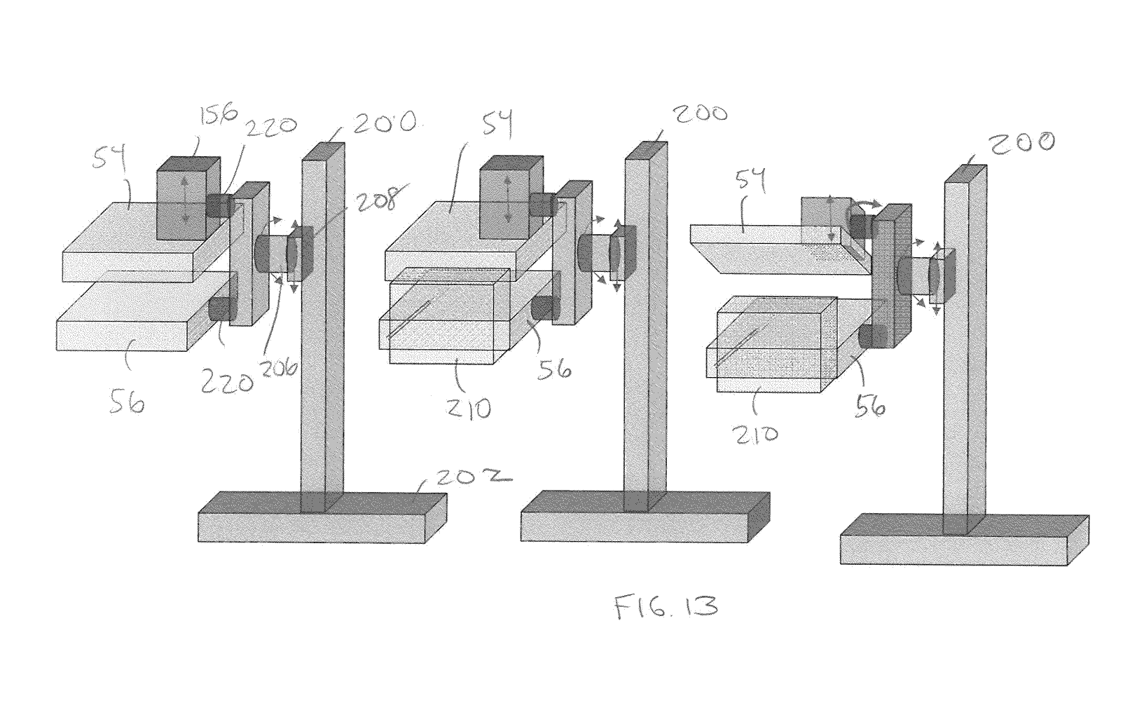

Different configurations of support structures may be provided, for example, as shown in FIGS. 11-14. As shown in FIG. 11, the gantry 102 may generally include a support 200 (illustrated as a vertical tower) on a base 202. A rotating arm 204 is coupled to the support 200 via a rotating portion 206 that allows the detectors 54 and 56 to rotate and a translating portion 208 that allows the detectors 54 and 56 to translate along the support 200. In this embodiment, a biopsy device 210 (which may include one or more biopsy components, such as the biopsy guiding device 162 as described herein) is mounted to one of the sides 205 of the detectors 54 and 56. However, the biopsy device 210 may be mounted to a front 207 of one of the detectors 54 and 56 as shown in FIG. 12. The biopsy device 210 may be mounted to any location as desired or needed, such as based on the orientation and position of the detectors 54 and 56. The biopsy device 210 also may be removable mounted or movable with respect to the detectors 54 and 56 to change the location of the biopsy device 210 with respect to the detectors 54 and 56. In some embodiments, the biopsy device 210 is mounted to the support 200 and movable relative to the support 200.

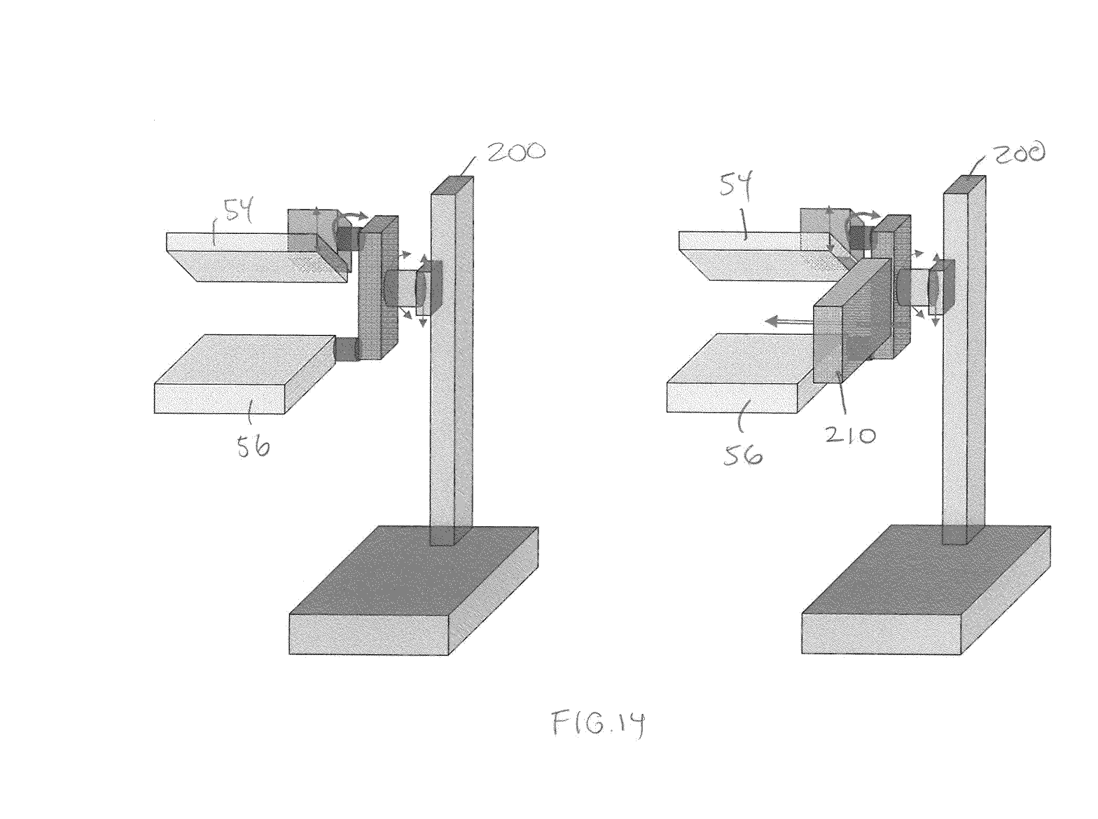

Additionally, as described herein, and as illustrated in FIGS. 13 and 14, the detectors 54 and 56 may be individually rotatable to independently tilt. For example, rotators 220 may couple the detectors 54 and 56 to the arm 204 that likewise may rotate. Also, in some embodiments, the biopsy device 210 may be used only in certain orientations or positions. Additionally, the gantry 102 also may include rotators in some embodiments to allow the detectors 54 and 56 to tilt towards and/or away from the body of the patient 180, for example, as shown in FIG. 6 or the patient 180 may be positioned differently, such as in front of or to the side of the gantry 102 to provide the different tilt configurations.

Accordingly, various embodiments provide one or more methods for imaging breasts and/or determining or approximating the location of a lesion within the breast. It should be noted that image information acquired by the various embodiments may be processed using any suitable method or process to, for example, approximate the depth or location of the lesion. For example, the depth of a lesion may be calculated using geometry, which may then be used to guide the direction of a biopsy needle.

The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid state drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), ASICs, logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the invention. The set of instructions may be in the form of a software program. The software may be in various forms such as system software or application software, which may be a tangible non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

What is claimed is:

1. A breast imaging system comprising:
a gantry;
a first nuclear medicine detector mounted to the gantry and having a multi-bore collimator coupled thereto; and
a second nuclear medicine detector mounted to the gantry and having a multi-bore collimator coupled thereto, wherein the first and second nuclear medicine detectors are configured to be independently titled with respect to a breast therebetween.

2. The breast imaging system of claim 1, wherein the first and second nuclear medicine detectors are configured to be titled transverse to the breast.

3. The breast imaging system of claim 1, wherein the first and second nuclear medicine detectors are configured to be titled one of towards or away from the breast in a plane perpendicular to a front of the breast.

4. The breast imaging system of claim 1, wherein the collimators are multi-bore, parallel-hole collimators.

5. The breast imaging system of claim 1, wherein the collimators are multi-bore, slanted-hole collimators.

6. The breast imaging system of claim 5, wherein the slanted-hole collimators are slanted obliquely to each other.

7. The breast imaging system of claim 5, wherein the slant angle for the bores of the collimator coupled to the first nuclear medicine detector is different than the slant angle of the bores of the collimator coupled to the second nuclear medicine detector.

8. The breast imaging system of claim 1, further comprising a biopsy device, wherein the first and second nuclear medicine detectors are configured to titled such that a distance between the first and second nuclear medicine detectors is greater at a location proximate to the biopsy device.

9. The breast imaging system of claim 1, wherein the first and second nuclear medicine detectors are configured to be titled in different directions or at different angles within respect to the breast.

10. The breast imaging system of claim 1, wherein the first and second nuclear medicine detectors are configured to translate and rotate independently.

11. The breast imaging system of claim 1, wherein the collimators are removably mounted to the first and second nuclear medicine detectors and further comprising a plurality of additional collimators, wherein at least some of the collimators with holes slanted at different angles than other ones of the collimators.

12. The breast imaging system of claim 1, wherein the first or second nuclear medicine detectors are rotatable with respect to the gantry such that the first or second nuclear medicine detector is positionable above the breast or below the breast.

13. The breast imaging system of claim 1, further comprising a biopsy guiding device configured to be mounted to the gantry.

14. A breast imaging system comprising:
a gantry;
a first nuclear medicine detector mounted to the gantry and having a multi-bore collimator coupled thereto; and
a second nuclear medicine detector mounted to the gantry and having a multi-bore collimator coupled thereto, wherein the first and second nuclear medicine detectors are configured to be independently titled with respect to a breast therebetween and the multi-bore collimators coupled to the first and second nuclear medicine detector have slanted bores, with the bores of the multi-bore collimator coupled to the first nuclear medicine detector slanted at a different angle than the bores of the multi-bore collimator coupled to the second nuclear medicine detector.

15. The breast imaging system of claim 1, wherein the bores of the multi-bore collimator coupled to the first nuclear medicine detector slanted at an oppositely oblique angle to the bores of the multi-bore collimator coupled to the second nuclear medicine detector.

16. The breast imaging system of claim 1, wherein the first and second nuclear medicine detectors are configured to rotate at least one of transverse to the breast or perpendicular thereto.

17. A method for breast imaging, the method comprising:
positioning a pair of opposing nuclear medicine detectors at an oblique angle to each other, wherein the nuclear medicine detectors include a multi-bore collimator coupled thereto;
maintaining a position of a breast between the nuclear medicine detectors;
acquiring nuclear medicine imaging data based on emissions from an agent injected into the breast; and
computing a depth of a lesion in the breast based on a relative difference in the location of the lesion imaged by each of the nuclear medicine detectors.

18. The method of claim 17, further comprising using the computed depth to guide a biopsy needle into the breast.

19. The method of claim 17, further comprising maintaining an asymmetrical position of the breast between the first and second nuclear medicine detectors.

20. The method of claim 19, further comprising positioning the first and second nuclear medicine detectors closer together at a location of the breast including a lesion therein.
